(19) United States
(12) Patent Application Publication
(10) Pub. No.: US 2011/0034920 A1
(43) Pub. Date: Feb. 10, 2011

(54) Variable dual mode tissue heating system controller

(76) Inventor: Htwe W. Naing, Reading, MA (US)

Correspondence Address: Htwe W. Naing, 20, Highland street, Reading, MA, 01867, US

(21) Appl. No.: 12/806,225

(22) Filed: Aug. 9, 2010

Related U.S. Application Data
(60) Provisional application No. 61/273,644, filed on Aug. 7, 2009.

(51) Int. Cl. A61B 18/12

(52) U.S. Cl. 606/41

(57) ABSTRACT

Present invention involves current mode and voltage mode which utilizes specific heat flux and heat transfer coefficient which when applied to the tissue, result in extra ordinarily effective.

### RELATED APPLICATIONS

The present application is a continuation-in-part application of U.S. provisional patent application Ser. No. 61/273644, filed Aug. 7, 2009, for VARIABLE DUAL MODE TISSUE HEATING SYSTEM CONTROLLER, by Htwe W. Naing, included by reference herein and for which benefit of the priority date is hereby claimed.

### FIELD OF THE INVENTION

The present invention relates to tissue heating system controller and, more particularly, to a variable voltage and current, as well as methods to accurately control series parallel combination resonance power inverter

### BACKGROUND OF THE INVENTION

Various kinds of typical single mode tissue heating systems have been developed and used today, such as Voltage mode (variable voltage and fixed current), current mode (fixed voltage and variable current). However, dual mode tissue heating system requires intelligent power conversion controller which integrates a micro controller or digital signal controller for a fully programmable and flexible solution. A micro controller or digital signal controller is a quite high noise sensitive device and difficult to implement for power conversion system.

Modern tissue heating systems include a resonance tank. The main purpose of the tissue heating system resonance tank is to provide maximum system performance with the least amount of current consumption and the best power efficiency possible. The resonance tank consists of the electronic passive components which require specific formula to generate clean signal. The passive components controlled by the source voltage and pulse current. In order to provide maximum performance to the system, it requires both source voltage and pulse current must be variable.

It would be advantageous to provide a variable voltage and variable current to tissue heating system.

It would also be advantageous to provide a series parallel resonance tank to tissue heating system.

### SUMMARY OF THE INVENTION

It is an object of the present invention to avoid the aforementioned and other disadvantages of conventional tissue heating system controller.

Accordingly, one object of the present invention is to provide an improved tissue heating systems accomplishing a variable voltage and variable current in the demanded series parallel resonance tank.

Another object of the present invention is to provide a tissue heating system for controlling the series parallel resonance tank in response to the alternative energy of system in order to improve system performance and reduce electrical energy consumption.

Furthermore, it is an object of the present invention is to provide a tissue heating system which can use a microcontroller or digital signal controller, and particularly, non analog controller which requires no higher component count.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a circuit diagram showing the operating circuit elements as well as their interconnections according to the present invention; and

FIG. 2 is a waveform diagrams, w1 to w6 are a series of curves view showing the voltage characteristics at various selected places throughout the circuitry of FIG. 1 and truth table of respective curves relationship.

For purposes of clarity and brevity, like elements and components will bear the same designations and numbering throughout the Figures.

### DESCRIPTION OF THE PREFERRED EMBODIMENT

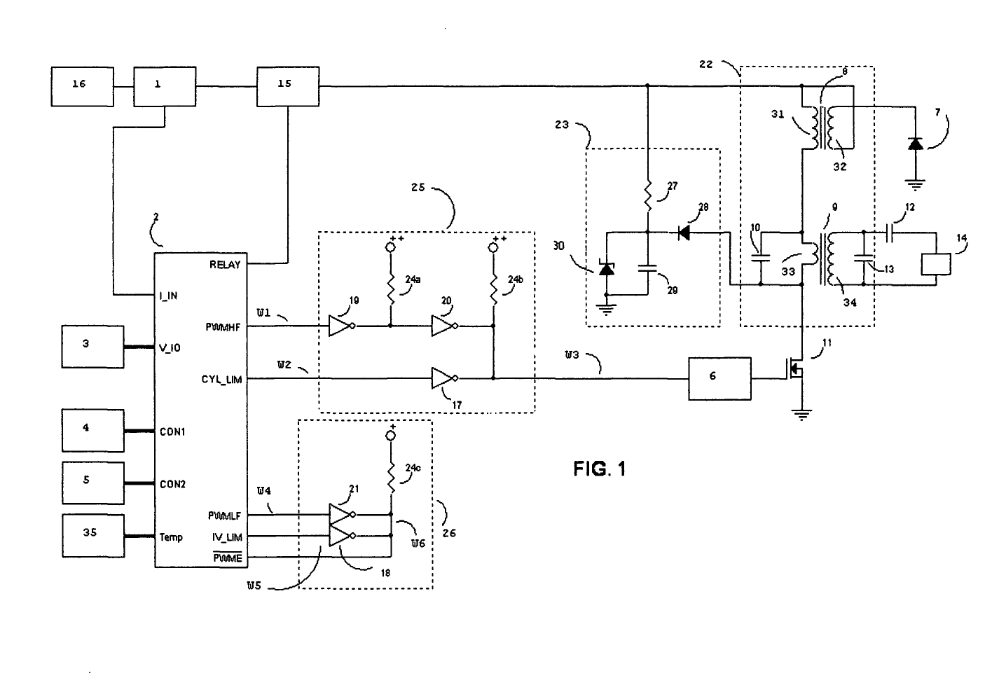

Referring now to the drawings, there is FIG. 1 shown an electrical schematic of a dual mode tissue heating system controller including a voltage variable boost converter 16 acting as a variable voltage source, current sensor 1 for sensing system current information, relay 15 for mechanical isolation between a voltage variable boost converter 16 and load 14, series parallel resonance tank 22, dc blocking capacitor 12 for blocking unwanted low frequency or dc voltage, MOSFET 11 for switching between system on time and off time, ultra fast diode 7 for guiding current flow direction of current limit transformer secondary winding 32 during MOSFET 11 off time, snubber circuitry 23 for dissipating unused energy (derived from leakage inductance of current limit transformer 8 and power transformer 9) during MOSFET 11 off time, mosfet driver 6 for driving proper MOSFET 11 gate voltage, open collector three buffer inverter circuitry 25, open collector two buffer inverter circuitry 26, micro controller 2 for controlling system energy and monitoring system information, voltage detector 3 for detecting voltage levels at several system locations, computer interface 4 for interfacing with computer, manual interface 5 for interfacing with manual controller and temperature sensor 35 for sensing system temperature.

Micro controller 2 includes V_IO interface for interfacing with voltage detector 3, CON1 interface for interfacing with computer interface 4, CON2 interface for interfacing with manual interface 5, Temp pin for sensing temperature from temperature sensor 35, I_IN pin for sensing system current information from current sensor 1, RELAY 15 pin for driving mechanical relay 15, PWMHF pin for generating high frequency pulse width modulation signal, CYL_LIM pin for generating maximum dutycycle of high frequency pulse width modulation signal, PWMLF pin for generating low frequency pulse width modulation signal, IV_LIM pin for generating over current or over voltage logic level state and PWME pin for detecting PWM signal enable or disable status.

Open collector two buffer inverter circuitry 26 includes low frequency PWM buffer 21 for buffering between PWMLF and PWME pin of micro controller 2, iv limit 18 for buffering between IV_LIM and PWME pin of micro controller 2 and pull up resistor3 24c for activating open collector inverter logic level.

Open collector three buffer inverter circuitry 25 includes high frequency PWM bufferl 19 for inverting logic level of micro controller 2 PWMHF pin output signal, high frequency PWM buffer2 20 for inverting logic level of high frequency PWM bufferl 19 output signal, dutycycle limit buffer inverter 17 for inverting logic level of micro controller 2 CYL_LIM pin output signal, pull up resistorl 24a and pull up resistor2 24b for activating open collector inverter logic level.

Snubber circuitry 23 includes snubber resistor 27 for dissipating and returning unused energy to source, snubber capacitor 29 for temporary storing unused energy, snubber diode 28 for guiding unused energy direction flow and over voltage protection diode 30 for limiting over voltage.

Series parallel resonance tank 22 includes current limit transformer 8, power transformer 9, primary resonance capacitor 10 for providing primary capacitive reactance (XCr) and secondary resonance capacitor 13 for providing secondary capacitive reactance (XCs).

Current limit transformer 8 includes current limit transformer primary winding 31 for limiting over current during MOSFET 11 on time and providing series inductive reactance (XLr), current limit transformer secondary winding 32 for dissipating unused energy during MOSFET 11 off time.

Power transformer 9 includes power transformer primary winding 33 for providing parallel inductive reactance (XLm) and power transformer secondary winding 34 for transferring energy to load 14 by turn ratio (N) of power transformer 9.

In order to maintain system stability and high efficiency, series parallel resonance tank 22 is defined by the following equations:

XLm˜2 ohm

XLr˜XLm*Pi

XCr˜2.5*XLm

XCr*N̂2˜XCs

Where

XLm=inductive reactance of power transformer primary winding 33.

XLr=inductive reactance of current limit transformer primary winding 31.

Pi=3.142.

XCr=primary capacitive reactance of primary resonance capacitor 10.

N=turn ratio of power transformer 9.

XCs=secondary capacitive reactance of secondary resonance capacitor 13.

Typically, all the fundamental parameters characterizing the system operative conditions are included in micro controller 2. These parameters are: voltage levels, current levels, isolation condition, user instruction, system temperature, PWM generators and emergency shutdown.

Operationally, the initial step in the system is micro controller 2 initialization sequence for deciding whether system is under normal operating condition or not. Micro controller 2 initialization sequence includes normal start up source voltage levels check for collecting initial source voltage value, snubber voltage level check for collecting initial snubber voltage value, current level check for collecting initial current value, system temperature level check for collecting system temperature value and compare with pre programmed respective EEPROM values to determine system condition. Mechanical relay 15 is switched from open to close position only when the system is normal operating condition. The next step is micro controller 2 normal operation sequence for operating system normal condition. Micro controller 2 normal operation sequence includes user interfacing for cooperating with user instructions through out the computer interface 4 or manual interface 5, variable source voltage level generator for generating user desired source voltage level controlled by micro controller 2 V_IO interface via voltage detector 3, variable current level generator for generating user desired pulse current level controlled by PWMHF pin and/or PWMLF pin of micro controller 2, over limit shutdown for terminating voltage generator and/or current generator via CYL_LIM or IV_LIM pins of micro controller 2 during exceeding user setting levels and power process for calculating system power consumption and system efficiency based on voltage, current and temperature values.

The arrangement of open collector three buffer inverter circuitry 25 provides for maintaining to turn off MOSFET 11 during the system power up sequence. For example, both PWMHF and CYL_LIM pins of micro controller 2 logic states high or low will generate logic low state on open collector three buffer inverter circuitry 25 output.

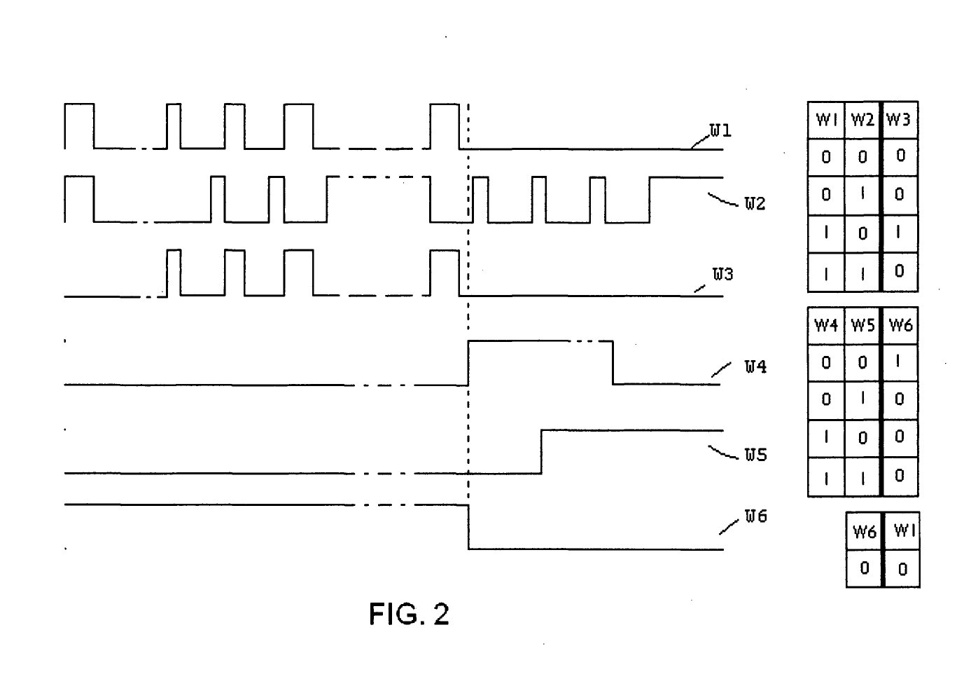

Referred to FIG. 2, W1 shown high frequency variable pulse width modulation signal generating by micro controller 2 PWMHF pin, W2 shown high frequency maximum pulse width limiting signal generating by micro controller 2 CYL_LIM pin, W3 shown high frequency pulse width modulation signal generating by open collector three buffer inverter circuitry 25 output, W4 shown low frequency variable pulse width modulation signal generating by micro controller 2 PWMLF pin, W5 shown over limit shutdown signal generating by micro controller 2 IV_LIM pin and W6 shown combination logic state output of PWMLF and IV_LIM pins which also controlled PWMHF pin logic state. Truth tables had shown respective curves relationship.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A variable dual mode tissue heating system controller for controlling tissue heating system in variable dual mode, comprising:
means for controlling system;
means for providing variable voltage; and
means for providing series parallel resonance.

2. The variable dual mode tissue heating system controller in accordance with claim 1, wherein said means for controlling system comprises a micro controller.

3. The variable dual mode tissue heating system controller in accordance with claim 1, wherein said means for providing variable voltage comprises a voltage variable boost converter.

4. The variable dual mode tissue heating system controller in accordance with claim 1, wherein said means for providing series parallel resonance comprises a series parallel resonance tank.

5. A variable dual mode tissue heating system controller for controlling tissue heating system in variable dual mode, comprising:
a micro controller, for controlling system;
a voltage variable boost converter, for providing variable voltage; and
a series parallel resonance tank, for providing series parallel resonance.

6. The variable dual mode tissue heating system controller as recited in claim 5, further comprising:
an open collector three buffer inverter circuitry, for providing output logic level low state while both inputs logic level are same states, directly connected to said micro controller.

7. A variable dual mode tissue heating system controller for controlling tissue heating system in variable dual mode, comprising:
a micro controller, for controlling system;
a voltage variable boost converter, for providing variable voltage;
a series parallel resonance tank, for providing series parallel resonance; and
an open collector three buffer inverter circuitry, for providing output logic level low state while both inputs logic level are same states, directly connected to said micro controller.
